(19) United States
(12) Patent Application Publication
(10) Pub. No.: US 2011/0112482 A1
(43) Pub. Date: May 12, 2011

(54) BREAKAWAY PEG TUBE

(76) Inventor: Russell Redd, St. George, UT (US)

(21) Appl. No.: 12/942,946

(22) Filed: Nov. 9, 2010

Related U.S. Application Data
(60) Provisional application No. 61/259,865, filed on Nov. 10, 2009.

(51) Int. Cl. A61M 39/10; B23P 17/04

(52) U.S. Cl. 604/175; 604/533; 29/428

(57) ABSTRACT

A break-way PEG tube uses a releasable connector between two tubing segments to facilitate release of a feeding portion of the PEG tube when pulled on to avoid accidental removal of a distal portion of the PEG tube from a patient's abdomen.

### PRIORITY

The present application claims the benefit of U.S. Provisional Application 61/259,865 filed on Nov. 10, 2009 which is herein incorporated by reference in its entirety.

### THE FIELD OF THE INVENTION

The present invention relates to a gastric feeding tube. More specifically, the present invention relates to a break-away PEG tube.

### BACKGROUND

There are a wide variety of situations in which a person has difficulty eating food or drinking water normally. A variety of medical conditions such as stroke, esophageal trauma, throat cancer and other disorders render it difficult for a person to properly swallow nourishment which they need. Additionally, people who are in comatose states, those suffering from advanced dementia or individuals who are simply unable to feed themselves need access to nutrition. However, these people are also at the greatest risk to aspirate food and/or liquids and suffer from aspiration pneumonia.

In enteral feeding and the like, a feeding tube may be inserted temporarily into a patient's stomach or intestine to allow food to be delivered to the patient while bypassing the physiological area of concern. This is commonly referred to as enteral nutrition. For short term situations, a tube may be inserted through a nasal passage and down into the patient's stomach or intestine. While nasoenteric feeding is desirable for short term feeding, the presence of a tube extending out of the nose is generally not desirable for long term feeding situations. The tube can be annoying to the patient and limits patient mobility.

For longer term feeding situations, it is common for a gastric feeding tube to be inserted through a small incision or stoma in the abdomen and extended into the stomach or intestine. The gastric feeding tube is often less irritating than a tube extending out of a nostril and provides a safe and effective method for delivering long-term enteral nutrition. The most common type of gastric feeding tube is the percutaneous endoscopic gastrostomy (PEG) tube.

A PEG tube is generally placed in an endoscopic procedure. The procedure may involve sedating a patient and then advancing an endoscope through the patient's mouth and esophagus and into the stomach or intestine. There are a variety of devices for determining location of the distal end of the endoscope or associated tools, such as the use of lights, magnetic or electronic detectors, etc.

Once the endoscope or associated tool is positioned in the stomach, a needle may be inserted through the abdomen and a lead line or suture attached to the needle is grasped by the endoscope or associated tool and the endoscope is withdrawn so that the lead line is drawn up through the esophagus and out the patient's mouth.

The lead line is then tied to the end of the PEG tube and the lead line is pulled back through the stoma in the patient's abdomen. This causes the PEG tube to be drawn down through the esophagus into the stomach and so that a portion of the PEG tube extends out of the stoma. A balloon or other retaining device prevents the PEG tube from being pulled completely out of the stoma under normal conditions. Once the PEG tube is properly positioned, it can be attached to a container of enteral feeding solution. The enteral feeding solution can be fed to the patient under the flow of gravity or can be driven and more precisely controlled by use of an enteral feeding pump.

Gastric feeding tubes may be suitable for long term use, and may last for up to six months. After prolonged usage, the stoma in the abdominal wall begins to scar or otherwise set in the tissues and the tube can be removed and replaced without the need to repeat the procedure through the esophagus. This can be done by inserting a PEG with an inflatable balloon through the stoma and then inflating the balloon to hold it in place.

One problem with the use of PEG tubes, especially with patients suffering dementia or otherwise being less than coherent, is the tendency for such patients to pull out the PEG tube. For example, a person suffering dementia may pull a PEG tube out if it is irritating him or her, not realizing that he or she has just severed the ability to receive nutrition. This is not uncommon in nursing homes and other long-term care facilities. If the PEG has been placed relatively recently, the entire procedure must often be repeated to place a new PEG tube. In that initial placement of a PEG tube can cost thousands of dollars, substantial time and expense are incurred returning the patient to the hospital, repeating the procedure and then returning the patient to the nursing facility. Moreover, some patients repeatedly pull out their PEG tubes. Thus, the costs can be significant.

Even when patients are cooperative, PEG tubes can be problematic. It is not uncommon for a person to accidentally cause the PEG tube to be pulled out by stepping on the feeding line or catching the PEG tube on something while being moved from one bed to another or from a bed to a wheel chair.

Accordingly, there exists a need for a break-away PEG tube that solves one or more of the problems herein described or that may come to the attention of one skilled in the art after becoming familiar with this disclosure.

### SUMMARY OF THE INVENTION

It is an object of the present invention to provide an improved break-away PEG tube.

According to one aspect of the invention, a break-away connection is provided for a PEG tube, such that a force on the PEG tube causes the connection to break rather than the tube being removed from the patient. Thus, a patient that may lack the understanding of the consequences of pulling on the PEG tube, or a person who inadvertently pulls on the PEG tube avoids damage to themselves, a procedure to replace the PEG tube and/or some risk of infection.

According to another aspect of the invention, the amount of force required for separation of the connection may be adjusted at the time of the insertion of the break-away PEG connection. This may be accomplished by inserting the joint to a specific depth and/or selecting a specific PEG joint. Thus, medical staff can determine the durability of the connection needed based on the risk of improper removal of the PEG tube.

These and other aspects of the present invention are realized in a break-away PEG tube as shown and described in the following figures and related description.

### BRIEF DESCRIPTION OF THE DRAWINGS

Various embodiments of the present invention are shown and described in reference to the numbered drawings wherein:

FIG. 1 shows a side, partially cut away, view of a break-away PEG tube system disposed in the abdominal wall of a patient;

FIG. 1A shows a side view of a break away PEG tube system used in conjunction with a feeding tube;

FIG. 2A shows a side view of a break-away PEG connector;

FIG. 2B shows a perspective view of the break-away PEG connector of FIG. 2A; and

FIG. 3 is a side elevational view of a plurality of designs and configurations of break-away PEG tube connectors.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity. Similarly, not every embodiment need accomplish all advantages of the present invention.

### DETAILED DESCRIPTION

The invention and accompanying drawings will now be discussed in reference to the numerals provided therein so as to enable one skilled in the art to practice the present invention. The drawings and descriptions are exemplary of various aspects of the invention and are not intended to narrow the scope of the appended claims.

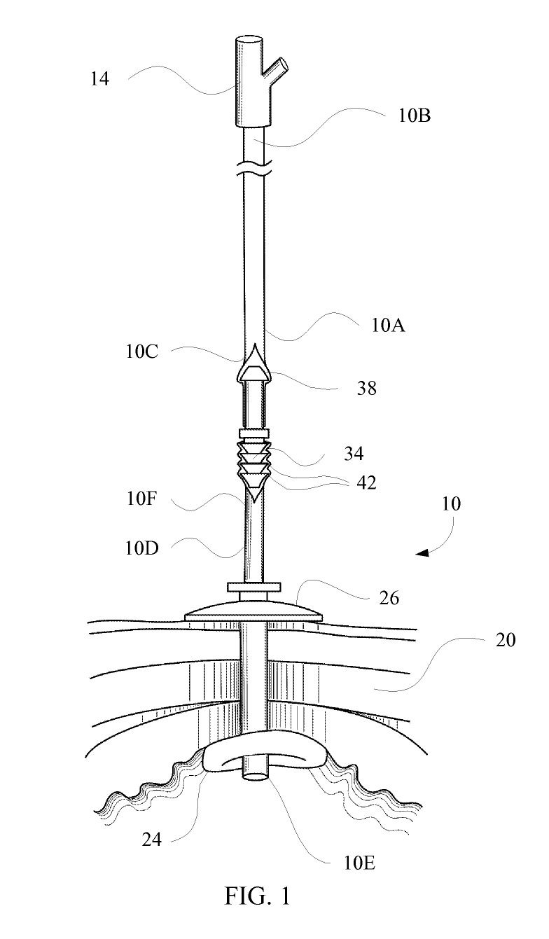

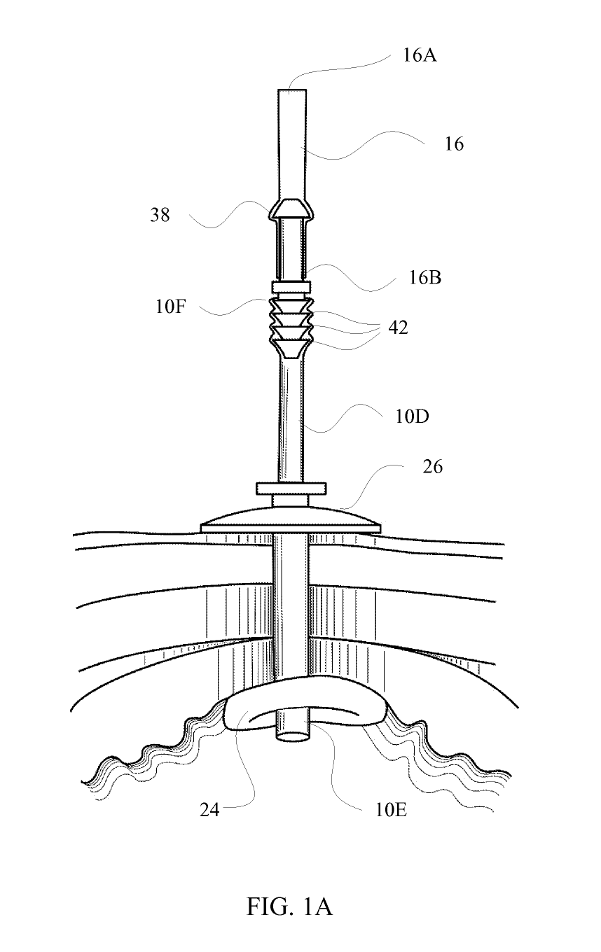

Turning now to FIG. 1, a side, partially cut-away view an endoscopic gastronomy system or PEG tube system is shown. A PEG tube, generally indicated at 10, is placed in the abdominal wall 20 of a patient so that a first, proximal portion 10A is disposed outside of the patient and extends between a first proximal end 10B and a second, distal end 10C. A distal portion 10D has a distal end 10E is disposed in the stomach or intestine. (It will be appreciated that modifications of the PEG tube, such as a PEGJ, which is designed to extend down into the intestine are included herein and are generally encompassed by references to the PEG tube unless expressly excluded).

Disposed at a first, proximal end 10B of the first, proximal portion 10A, the PEG tube 10 may include a cap 14 into which a connector for a feeding solution container and feed line are attached to provide nutrition. Such attachment mechanisms are well known in the art and are not discussed in detail herein. As shown in FIG. 1A, the first, proximal portion 10A could be replaced directly with a feed line 16 connected directly to an enteral feeding supply, the feeding line 16 may include a first, proximal end 16A attached to the feeding supply and a second, distal end 16B connected to the PEG tube 10.

The distal portion 10D of the PEG tube 10 may include an internal (i.e. inside the patient) retaining device 24 for holding the distal end 10E of the distal portion of the PEG tube inside the stomach, etc., while allowing a more proximal portion of the distal portion 10D to extend through the abdominal wall 20 and past an exterior (i.e. outside the patient) retaining device 26 to terminate in a proximal end 10F. The internal retaining device 24 may be an inflatable balloon, or other retention mechanism such as are known in the art or may later be developed. The external retaining device 26 covers the stoma or hole in the abdominal wall 20 through which the PEG tube extends to minimize or prevent the movement of the tube relative to the abdominal wall. Such devices as a bolster or other retainers known in the art can be used for the external retaining device.

In accordance with one aspect of the present invention, distal portion 10D of the PEG tube 10 may be cut or formed so that it extends between 1 inch and 5 inches (typically 1-3 inches) from the external retaining device 26 or patient's abdominal wall 20 when installed, and then has the distal portion terminate at a proximal end 10F. A break away PEG connection 34 (shown in the cut-away portion of FIG. 1) may be mounted in the first, proximal end 10F of the distal portion 10D and in the second, distal end 10C of the proximal portion to connect the two together to form the PEG tube 10. The break-away PEG connector 34 connects the two segments and has a bore extending from an open first end disposed in the distal end 10C of the proximal portion 10A, to an open second end in the proximal end 10F of the distal portion 10D to allow enteral nutrition to be delivered through the PEG tube 10.

The break-away PEG connector 34 couples the feeding tube or proximal portion 10A of the PEG tube 10 to the distal portion 10D of the PEG tube, allowing the flow of nutrition through the PEG tube and into the patient. The tube may also be used for withdrawing stomach fluids if needed for testing, etc.

Occasionally, patients pull out conventional PEG tubes in discomfort or unintentionally as they attempt to move. However, the break-away PEG connection 34 is configured to disconnect the distal portion 10D and the feeding tube or proximal portion 10A at a lower force threshold than is normally required to pull the balloon 24 or other retaining device on the distal portion 10D out of the patient. Thus, the connector 34 may form a sacrificial connection with at least one of the tubes 10C, 10D and thus is configured to prevent the removal of the endoscopic gastronomy system from the stomach aperture of the patient.

The break-away PEG tube 10 may provide resistance from separation. This is typically done by projections 38, 42 (which may include annular or circumferential ribs, linear projections, etc.) on the connector 34 being inserted into the tubing of the PEG tube (i.e., inserting the projections 42 into the proximal end 10F of the distal portion 10D and the projections 38 into the distal end 10C of the proximal portion 10A) and resisting removal from the tubing. It will be appreciated that the size of the projections will depend on the inner diameter of the tubing which forms the PEG tube 10 and on its radial expansivity. However, the projections 38, 42 will typically be between 0.01 inches and 0.5 inches, with a more specific length being dependent on the tubing.

If a force is applied to the PEG tube 10 exceeds a desired threshold, the break-away PEG connector 34 may allow the PEG tube distal portion 10D and feeding tube or proximal portion 10A to separate from one another. This releasable connection prevents a patient from tugging on the PEG connection with sufficient force to remove the PEG tube or damage the patient. This, in turn, reduces the need for surgery to replace the PEG tube, remediation of damage to the patient stomach, abdomen and other tissues, resulting in potential pain, cost and wasted time.

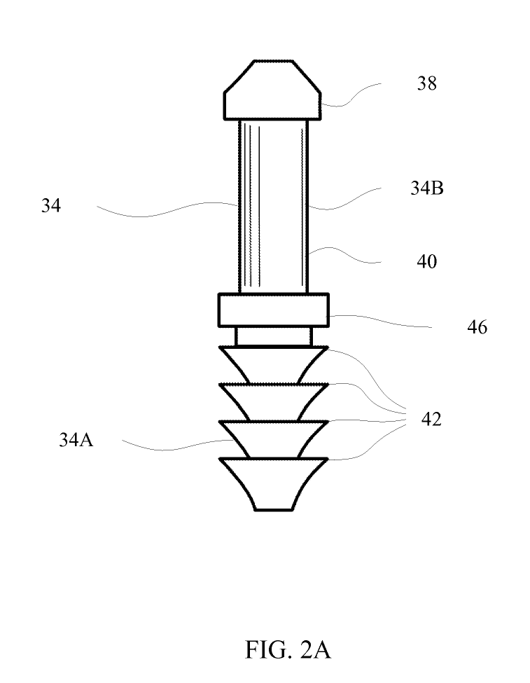

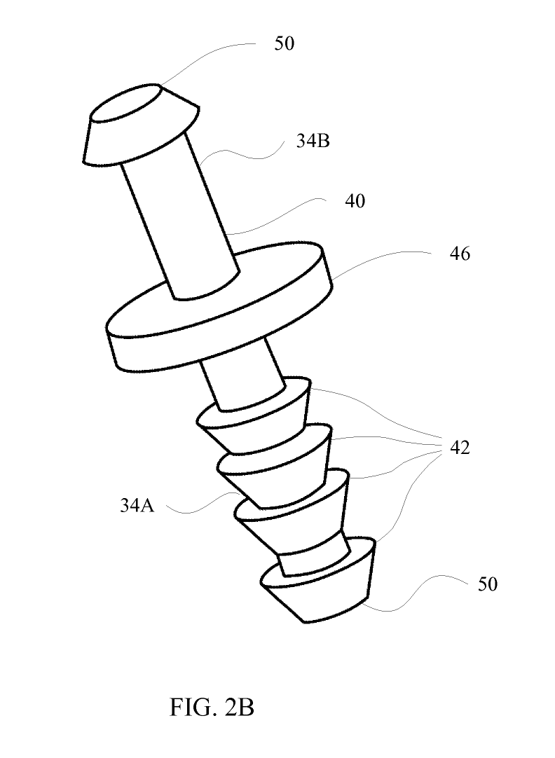

FIGS. 2A and 2B show a side view and a perspective view of a break-away PEG tube connector 34, respectively. The PEG connector 34 may be formed from a variety of materials, typically including biocompatible plastics, vinyls, etc. The PEG tube connector 34 has a body 40 which may be formed from a single piece or may be composed of multiple pieces. A plurality of projections 38 and 42 disposed along portions of the connector body 40 extend outwardly to engage the tubing which forms the proximal and distal portions 10A, 10D of the PEG tube, to releasably engage and hold the tubing portions together under normal conditions. A central portion of the PEG tube connector 34 body may have a disc or flanges 46 formed thereupon, to form a stop. The stop 46 may prevent a connected tube from advancing farther than the stop. The PEG connector 34 may also include a central bore (50, FIG. 2B), allowing matter to pass from one end to the other. Typically, the central bore 50 is close in diameter to the diameter of the tubing 10A and 10D so that the connector provides minimal interference with flow of the enteral feeding solution passing therethrough.

The PEG connector 34 may be configured to be biased to remain in one tube and not the other when the proximal and distal portions of the PEG tube become disengaged. For example, it may be desirable to have the PEG connector 34 remain with the patient. Thus, the distal or patient side 34A of the PEG connector 34 may include multiple and/or larger protrusions, such as flanges, ridges, bumps, ribs, spiral, high friction engagement surfaces, or other extensions 42 from the connector body. The feeding or proximal side 34B may include a single and/or smaller protrusion. The net effect of multiple and/or larger protrusions may be that the PEG connector has a higher resistance to being pulled from the distal portion 10D of the PEG tube 10 than from the feeding side or proximal portion 10A. Thus, a force would typically first overcome the feeding side resistance. This would result in the removal of the feeding tube or proximal portion 10A of the PEG tube from the PEG connector 34, but the patient tube or distal portion 10D would remain connected on the PEG connector, with the distal portion 10D of the PEG tube disposed in the abdomen.

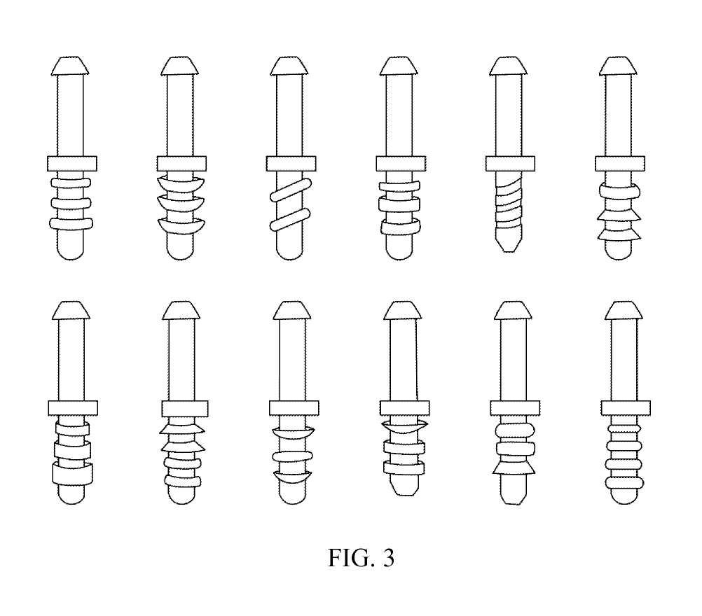

Turning now to FIG. 3, a plurality of designs and configurations of break-away PEG connectors 34 are shown. There is a break-away PEG tube configured to include a plurality of protrusions. The plurality of protrusions include sizes of members and/or spacing that are different in various embodiments and vary according to the particular requirements of the PEG tube. As illustrated in FIG. 3, the protrusions may be of a different shape, different number, have different spacing and/or may or may not be symmetrical about the long axis of the tube. Furthermore, the plurality of protrusions may include a spiral configuration, a graduated configuration, a levered configuration, and/or a textured configuration, and still perform its intended function.

The various geometries may include a graduated square rib, a double rounded rib and angled disc, an alternating angled disc and rib, a series of ribs followed by an angled disc, an angled disc followed by a series of ribs, a graduated rounded rib, a series of rounded ribs, a series of angled discs, a spiral rib, a series of square ribs, a spiral channel, and a series of angled discs followed by a rounded rib. Other geometries may include knobs or fins.

However, it should be recognized that FIG. 3 does not provide an exhaustive list of embodiments. The ideas and components may be further mixed and/or matched to create more PEG connectors. Furthermore, while it has been discussed and/or shown that the PEG connector may be symmetrical, it may also be asymmetrical. In one embodiment, the protrusions extend between ½ and ⅞ around the circumference of the central body of the PEG connector. The protrusions serve to push a flat portion of the central body (without protrusions) to the side of the tube.

Similarly, while many embodiments shown have one or three protrusions per side, there may be more or less protrusions per side. The embodiments shown are just examples given to demonstrate a possibility of multiple or single protrusions per side. Other numbers have been considered, but the current examples are shown for clarity.

For example, although the illustrated break-away tube of FIG. 2A shows a side view of four equally spaced saw-tooth protrusions it is understood that the protrusions may be of a different shape, different number, have different spacing and may or may not be symmetrical about the long axis of the tube.

Additionally, although the figures illustrate particular sizing and spacing, it is understood that sizes of members and/or spacing may be different in various embodiments and may vary according to the particular requirements filled by the PEG tube.

It is also envisioned that the break-away tube connector 34 may include one or more features configured to alter a force requirement before break-away, such as, but not limited to, graduated protrusions, spiral protrusions, a levered protrusion, and/or a set of progressively shaped/textured/material protrusions such that insertion of the break-away tube is possible in a plurality of manners or degrees and that the plurality of manners or degrees results in varied break-away force thresholds or friction effects or break-away mechanics. Accordingly, a user may be enabled to insert and/or operate the break-away tube and connector in a manner that permits the user to select between a set or continuum of coupling configurations having disparate break-away points. Thereby the same device configuration may be utilized for a variety of patients having a variety of issues that may be addressed by the break-away PEG.

For example, in one embodiment, the feeding tube side of the break-away PEG connector may include a set of ribs increasing in size from a smaller rib at the tip or proximal end to a larger rib near the center of the connector. Between or on each rib may be labeled with an estimated force if that rib is chosen. A health care provider may then select the force by placing the tube onto the break-away PEG connector 34, ensuring the tube overlaps the rib with the desired force requirement.

Finally, it is envisioned that the components of the device may be constructed of a variety of materials, including but not limited to plastics, rubber, ceramic, wood, metal, composites, and combinations thereof.

There is thus disclosed an improved break-away PEG tube. It will be appreciated that numerous changes may be made to the present invention without departing from the scope of the claims.

What is claimed is:

1. An endoscopic gastronomy system, comprising:
a proximal tube portion;
a distal tube portion; and
a break-away tube connector configured to couple the proximal tube portion to the distal tube portion such that applying a given force to the proximal tube portion separates the proximal tube portion from the distal tube portion, to prevent the removal of the system from the stomach aperture of the patient.

2. The endoscopic gastronomy system of claim 1, wherein the distal tube portion comprises a retaining device disposed toward a distal end of the distal tube portion, the retaining device comprising at least one of a balloon and a bolster.

3. An endoscopic gastronomy system, comprising:
a first tube, the first tube exiting a patient, the first tube further comprising a first end, the first end being external to the patient;
a second tube, the second tube further comprising a first end;
a tube connector, the tube connector joining the first end of the first tube and the first end of the second tube, the tube connector further comprising:
a body having a distal portion and a proximal portion and a bore extending therebetween, the distal portion comprising at least one projection extending outwardly and at least one projection engaging a first tube portion and the proximal portion comprising at least one projection extending outwardly therefrom, the at least one projection of the proximal portion engaging a second tube portion, the tube connector holding the first tube portion to the second tube portion.

4. The endoscopic gastronomy system of claim 3, wherein the projections of the distal portion of the body of the tube connector and the projections of the proximal portion of the body of the tube connector provide a friction such that a force required to pull the first tube portion from the distal portion of the body of the tube connector exceeds a force required to pull the second tube portion from the proximal portion of the body of the tube connector.

5. The endoscopic gastronomy system of claim 3, wherein the at least one projection of the distal portion and the at least one projection of the proximal portion provide a friction such that a force required to separate the tube connector from the first or second tube is less than would be required to remove the first tube from the patient.

6. The endoscopic gastronomy system of claim 3, wherein the first tube extends outside the patient body between 1 and 5 inches.

7. The endoscopic gastronomy system of claim 3, wherein the first tube extends about 3 inches outside the patient body.

8. A method of forming a breakaway PEG connection, the method comprising:
affixing a PEG tube to a patient;
making the PEG tube roughly a pre-determined length outside the patient;
selecting a PEG connector for a connection between the PEG tube and a feeding tube;
inserting a PEG connector into the PEG tube; and
connecting the feeding tube to the PEG connector.

9. The method of claim 8, wherein the selecting step further comprises the step of choosing a PEG connector based on the force required to break a connection between the PEG tube and the feeding tube.

10. The method of claim 8, wherein the affixing a PEG tube to a patient step further comprises the step of cutting the PEG tube to a length of between 1 and 5 inches outside the patient body.

11. The method of claim 8, wherein the affixing a PEG tube to a patient step further comprises the step of cutting the PEG tube to a length of about 3 inches outside the patient body.

12. A breakaway PEG connector comprising:
an elongated body with an elongated axis and a first end and a second end;
a bore extending along the elongated axis; and
one or more protrusions on the body.

13. The break away PEG connector of claim 12, wherein the protrusions are selected from the group of a rib, angled disc, a spiral rib, a spiral channel, a channel, knobs, and fins.

14. The break away PEG connector of claim 12, wherein the one or more protrusions further comprise a first group of protrusions on the first end and a second group of protrusions on the second end.

15. The break away PEG connector of claim 14, wherein the first group of protrusions are graduated from a center portion of the body to the first end.

16. The break away PEG connector of claim 14, wherein the first group of protrusions are

17. The break away PEG connector of claim 14, wherein the first group of protrusions comprises one or more angled discs.

18. The break away PEG connector of claim 17, wherein the second group of protrusions comprises an angled disc near the bore entrance.

19. The break away PEG connector of claim 12, further comprising a tube stop between the first end and the second end.

20. The break away PEG connector of claim 19, wherein the tube stop is disc shaped.

21. The break away PEG connector of claim 20, wherein the tube stop diameter is larger than an inner diameter of a tube placed on the PEG connector.
